(19) United States
(12) Patent Application Publication
(10) Pub. No.: US 2012/0330333 A1
(43) Pub. Date: Dec. 27, 2012

(54) DEVICE FOR REMOVING ACROCHORDONS

(75) Inventors: Staffan Sundström, Helsingborg (SE); Chris Anderson, Linkoping (SE); Michail Llias, Linkoping (SE); Carina Hartleb Fredriksson, Linkoping (SE); Poul Leo Anker, Birkerod (DK)

(73) Assignee: TAGAWAY DEVICES APS, Rungsted Kyst (DK)

(21) Appl. No.: 13/575,587

(22) PCT Filed: Feb. 1, 2011
(86) PCT No.: PCT/IB2011/000265

§ 371 Date: Aug. 30, 2012

Related U.S. Application Data
(60) Provisional application No. 61/300,332, filed on Feb. 1, 2010.

(51) Int. Cl. A61B 17/122; A61B 17/128

(52) U.S. Cl. 606/157

(57) ABSTRACT

A device (10) for occlusion of a skin tag (1) projecting from a skin area including a base member (11) having an upper side (12), an underside (13), and an aperture (14) for enclosing the skin tag, a first pressure member (16) connected to the base member at one end (16a) of the first pressure member, a first occlusion member (17) connected to an other end (16b) of the first pressure member for applying an occlusion force to the enclosed skin tag, a second pressure member connected to the base member at one end of the second pressure member, a second occlusion member connected to an other end of the second pressure member for applying an occlusion force to the enclosed skin tag, and a gap (18) between the first and second occlusion members configured to apply an occlusion force sufficient to occlude blood flow to the skin tag, and a method of using the device are disclosed.

### CROSS-REFERENCE TO RELATED APPLICATION

The present application is based upon and claims the benefit of priority from the prior U.S. Provisional Application Ser. No. 61/300,332, filed on Feb. 1, 2010, the entire contents of which are incorporated herein by reference.

### FIELD OF THE INVENTION

The present invention relates to a device for removing acrochordons, and a method of using the device for removing acrochordons.

### BACKGROUND

The occurrence of potentially life-threatening malignant neoplasia in the skin focuses attention on all departures from the skin's normal appearance. Since most parts of the skin are accessible for inspection, there is an opportunity for early detection, by the patient, of potentially dangerous neoplasms such as malignant melanoma, squamous cell cancer and a long list of other rare neoplasms. Great effort is spent in educating the population to seek medical advice early. The vast majority of skin lesions are, however, benign. Thus the majority of individuals seek advice for lesions for which there is no medical need for treatment. The patient may, however, desire removal of the lesions for cosmetic reasons.

Common benign skin lesions include verruca, seborrhoic keratoses, fibromas, histiocytomas (dermatofibromas), nevi and skin tags. A skin tag is a small outgrowth of epidermal and dermal tissue, one to several millimeters in size, usually flesh-colored and pedunculated. Skin tags display a variety of shapes, sizes and histology, as shown in FIG. 1, and are described by alternative names, of which acrochordon is the most accepted. The common sites for skin tags are the neck, axillae, trunk and groin, but they can occur all over the body. Larger lesions are often fibrosed nevi.

Histologically, as shown in FIG. 2, a skin tag 1 may show localized hyperplasia of the dermis 3 with loosely arranged collagen fibers and dilated capillaries 4 and lymphatic vessels 5, indicating that a skin tag 1 is softer, more elastic, and about as vascularized as the surrounding skin area 2. The epidermis 6 can be slightly hyperplastic.

The cellular characteristics of the skin tag may be important from the point of view of removal by pressure at the base. For example, for a floppy pedunculated skin tag in which the connective tissue stroma is thin and loose with few cells, externally exerted pressure would result in little resistance from the tissue, and the vasculature would easily collapse. These lesions tend to be smaller in size.

However, skin lesions in which the presence of nevus cells and a firm connective tissue network may protect the vasculature at the base from pressure may often be larger, broader in base and “dome shaped,” and exert more resistance to compression of the base.

A complete occlusion of the vasculature at the base of a skin tag could be expected to result in a necrosis of the skin tag. When occlusion of the base is incomplete, a degree of inflammation may be expected, which inflammatory response, by causing swelling, may enhance the occlusive effect.

The current medical treatment of skin tags involves clipping or shaving the lesion at the base, cryotherapy, or diathermy. Cryotherapy is perceived as inexpensive and does not require anesthesia, but there may be a number of drawbacks. For example, if cryotherapy were not targeted, it may result in damage to healthy tissue, it may be painful, and there may be a risk of blistering, scarring, and pigmentation changes. Alternatively, patients can undertake “self-treatment” by traumatizing the most accessible lesions in different ways, including tying off the lesions with a sewing thread.

Patients may be motivated to the removal of skin tags, but the health care infrastructure is often less interested because skin tags are benign and the cost of clinical intervention can be hard to justify, regardless of the psychological impact on the patient. In this regard, skin anomalies such as skin tags may strongly affect the patients' quality of life. For example, even slight changes in the skin may result in adjustments to social life, relationships, and even the ability to carry out daily tasks.

### SUMMARY

Accordingly, there is a need for a device for removing acrochordons safely, quickly and discreetly, which may be easily utilized by a patient with minimal pain, blistering, scarring, pigmentation changes or other potential drawbacks. Further, there is a need for a device that may be used by patients without significant professional medical intervention.

In a non-limiting embodiment of the present invention, a device for occlusion of a skin tag projecting from a skin area comprises a base member having an upper side, an underside, and an aperture for enclosing the skin tag; a first pressure member connected to the base member at one end of the first pressure member; a first occlusion member connected to an other end of the first pressure member for applying an occlusion force to the enclosed skin tag; a second pressure member connected to the base member at one end of the second pressure member; a second occlusion member connected to an other end of the second pressure member for applying the occlusion force to the enclosed skin tag; and a gap between the first and second occlusion members configured to apply the occlusion force sufficient to occlude blood flow to the skin tag.

In an alternative non-limiting embodiment of the invention, the underside of the base member includes an adhesive for fixing the base member to the skin area.

In an alternative non-limiting embodiment of the invention, the base member is one of rectangular, square, circular, oval and elliptical.

In an alternative non-limiting embodiment of the invention, the other end of the first pressure member faces the other end of the second pressure member.

In an alternative non-limiting embodiment of the invention, the first occlusion member, the first pressure member, and a portion of the base member adjacent to the first pressure member are formed in the shape of one of a “Z” and a “W”.

In an alternative non-limiting embodiment of the invention, the second occlusion member, the second pressure member, and a portion of the base member adjacent to the second pressure member are formed in the shape of one of a “Z” and a “W”.

In an alternative non-limiting embodiment of the invention, a pair of first pressure members is connected to the first occlusion member.

In an alternative non-limiting embodiment of the invention, a pair of second pressure members is connected to the second occlusion member.

In an alternative non-limiting embodiment of the invention, the first pressure member includes a plurality of first pressure members, and the first occlusion member includes a plurality of first occlusion members.

In an alternative non-limiting embodiment of the invention, the second pressure member includes a plurality of second pressure members, and the second occlusion member includes a plurality of second occlusion members.

In an alternative non-limiting embodiment of the invention, the first occlusion member faces the second occlusion member for jointly applying the occlusion force to the enclosed skin tag.

In an alternative non-limiting embodiment of the invention, the occlusion force sufficient to occlude blood flow to the skin tag is in a range of about 5 g to about 200 g.

In an alternative non-limiting embodiment of the invention, the occlusion force sufficient to occlude blood flow to the skin tag is in a range of about 8 g to about 109 g.

In an alternative non-limiting embodiment of the invention, the gap between the first and second occlusion members is in a range of about 0.1 mm to about 4.0 mm.

In an alternative non-limiting embodiment of the invention, the gap between the first and second occlusion members is in a range of about 0.2 mm to about 2.0 mm.

In an alternative non-limiting embodiment of the invention, the device is one of flat and disc-shaped.

In yet another non-limiting embodiment of the present invention, a method of using a device for occlusion of a skin tag projecting from a skin area, the device comprising a base member having an upper side, an underside, and an aperture for enclosing the skin tag, a first pressure member connected to the base member at one end of the first pressure member, a first occlusion member connected to an other end of the first pressure member, a second pressure member connected to the base member at one end of the second pressure member, a second occlusion member connected to an other end of the second pressure member, and a gap between the first and second occlusion members configured to apply an occlusion force sufficient to occlude blood flow to the skin tag, comprises applying compression to the device, whereby the gap between the first and second occlusion members is enlarged; enclosing the skin tag within the enlarged gap between the first and second occlusion members; and releasing the compression on the device, whereby the occlusion force is applied to the skin tag via the first and second occlusion members and the first and second pressure members.

In an alternative non-limiting embodiment of the invention, the compression is manually applied by a user's thumb and finger.

In an alternative non-limiting embodiment of the invention, the device is applied with one hand of a user.

In an alternative non-limiting embodiment of the invention, the method further comprises adhering the base member to the skin area via an adhesive.

In an alternative non-limiting embodiment of the invention, the occlusion force sufficient to occlude blood flow to the skin tag is in a range of about 5 g to about 200 g.

In an alternative non-limiting embodiment of the invention, the occlusion force sufficient to occlude blood flow to the skin tag is in a range of about 8 g to about 109 g.

Other features and aspects of the present invention will become more fully apparent from the following brief description of the drawings, the detailed description of the non-limiting embodiments, the appended claims and the accompanying drawings.

### BRIEF DESCRIPTION OF THE DRAWINGS

FIG. 1 illustrates a variety of types of skin tags.

FIG. 2 illustrates a histology of a skin tag.

FIG. 3 illustrates a first exemplary embodiment of a device according to the present invention.

FIG. 4 illustrates a second exemplary embodiment of a device according to the present invention.

FIG. 5 illustrates a schematic flow diagram of a method of using a device according to the present invention.

### DETAILED DESCRIPTION OF THE EMBODIMENTS

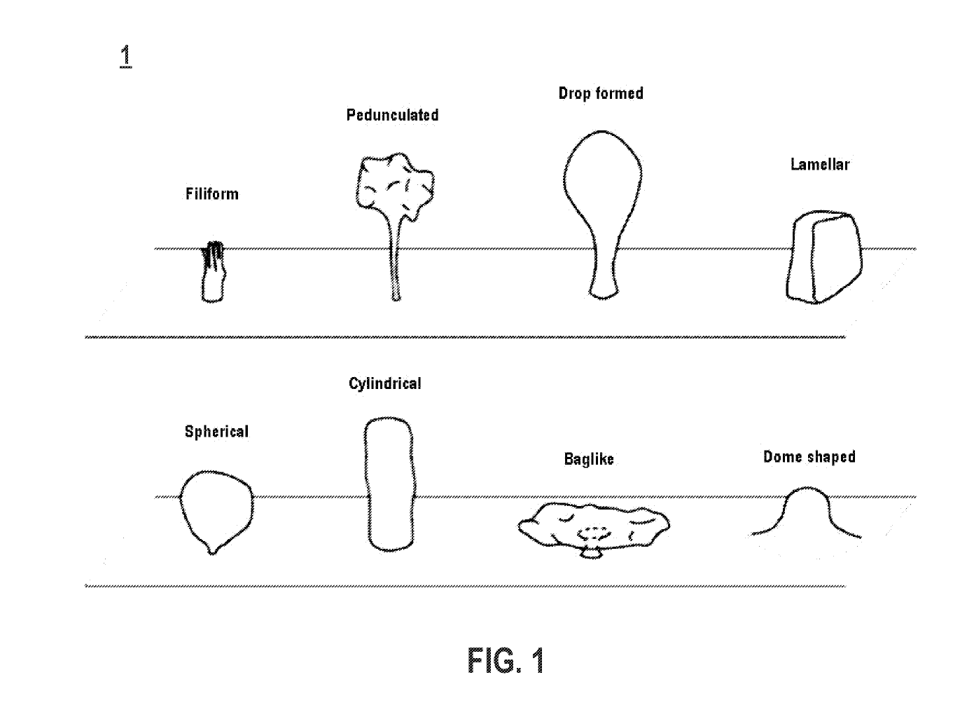

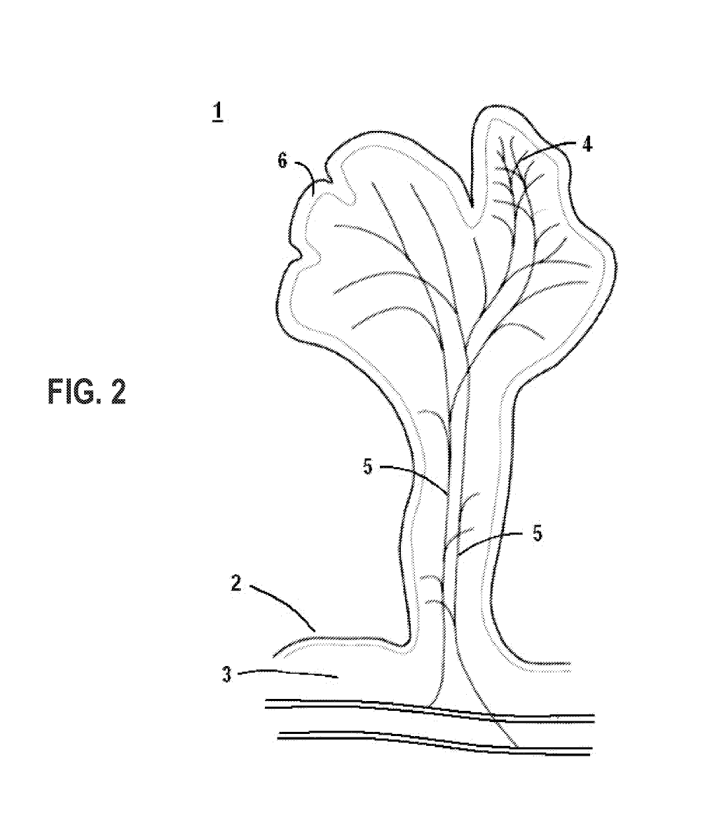

FIG. 1 illustrates a variety of types of skin tags, such as filiform, pedunculated, drop formed, lamellar, spherical, cylindrical, baglike, and dome shaped. Skin tags may exhibit a variety of shapes, sizes, histology (shown in FIG. 2), and other characteristics. The devices according to the present invention may be used to occlude, or occlusively remove, one or more of the variety of skin tags.

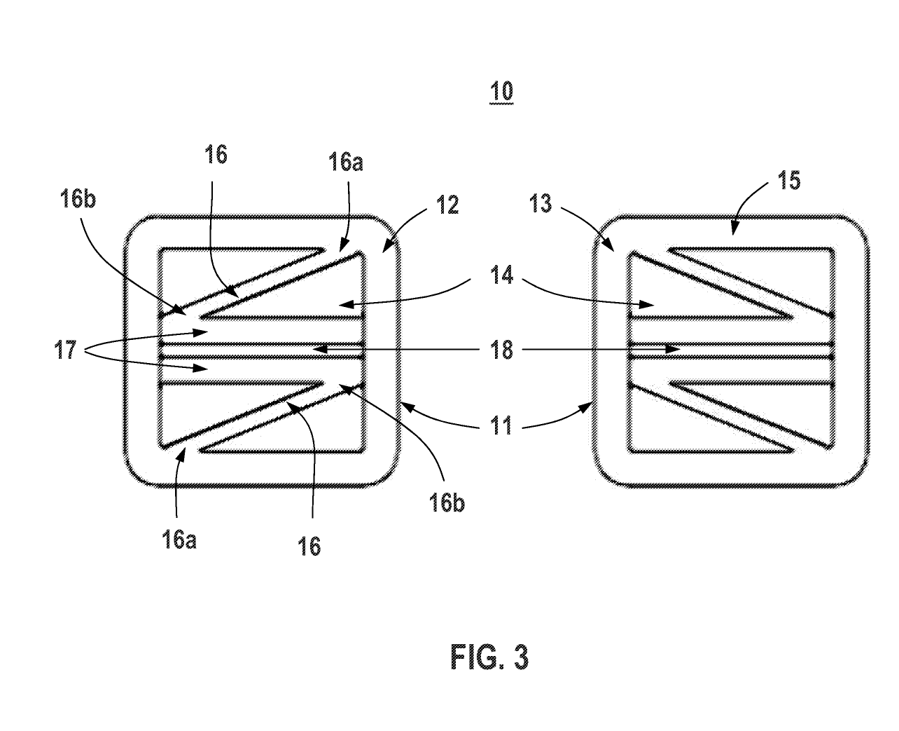

FIG. 3 illustrates a first exemplary embodiment of a device 10 for occlusion of skin tags according to the present invention. The device 10 may be flat or disc-shaped. The device 10 may include a base member 11 having an upper side 12, an underside 13, and an aperture 14 for enclosing a skin tag 1 on a skin area 2. The left side of FIG. 3 shows the upper side 12 of the device 10, and the right side of FIG. 3 shows the underside 13 of the device 10. The base member 11 may include a variety of shapes, including rectangular, square, circular, oval, elliptical and others. The underside 13 of the base member 11 may include an adhesive 15 in order to affix the base member 11 to the skin area 2. The adhesive 15 may cover all or only a portion of the underside 13 of the base member 11.

Situated within the aperture 14 of the base member 11, the device 10 may also include a pressure member 16 connected to the base member 11 at one end 16a of the pressure member 16. Preferably, the device 10 may include at least two pressure members 16 that are each connected to the base member 11 at one end 16a of each pressure member 16. The other end 16b of each pressure member 16 may face an opposing pressure member 16.

Also situated within the aperture 14 of the base member 11, the device 10 may also include an occlusion member 17 connected to the other end 16b of each pressure member 16. Preferably, the device 10 may include at least two occlusion members 17 that are each connected to the other end 16b of a pressure member 16, such that each occlusion member 17 faces an opposing occlusion member 17. Additionally or alternatively, the device 10 may also include a pair of pressure members 16 connected to each occlusion member 17. Moreover, the device 10 may also include a plurality of pressure members 16 and a plurality of occlusion members 17. Each occlusion member 17 may not be directly connected to the base member 11, such that each occlusion member 17 may move within the aperture 14 relative to the base member 11.

The opposing occlusion members 17 may be separated from each other by a gap 18, which may be in the range of about 0.1 mm to about 4.0 mm, preferably about 0.2 mm to about 2.0 mm. The opposing occlusion members 17 may jointly apply pressure to an enclosed skin tag 1 sufficient to occlude blood flow to the skin tag 1. The occlusion force sufficient to occlude blood flow to the skin tag 1 may be in the range of about 5 g to about 200 g, preferably about 8 g to about 109 g. Below about 5 g of force, the blood flow to the skin tag 1 may not be sufficiently occluded, and above about 200 g of force, the occlusion members 17 may cut the skin tag 1 off the skin area 2, instead of occluding blood flow to the skin tag 1. Preferably, the occlusion members 17 may apply pressure to a base of the enclosed skin tag 1, such that no portion of the skin tag 1 remains after occlusion and removal of the skin tag 1.

As shown in FIG. 3, each occlusion member 17, pressure member 16, and a portion of the base member 11 adjacent to the pressure member 16 may be formed in the shape of a “Z”. Based on the shape, size, thickness, material properties, and other characteristics of the pressure member 16 and occlusion member 17, each pressure member 16 and occlusion member 17 may elastically deform to apply an appropriate occlusion force to an enclosed skin tag 1.

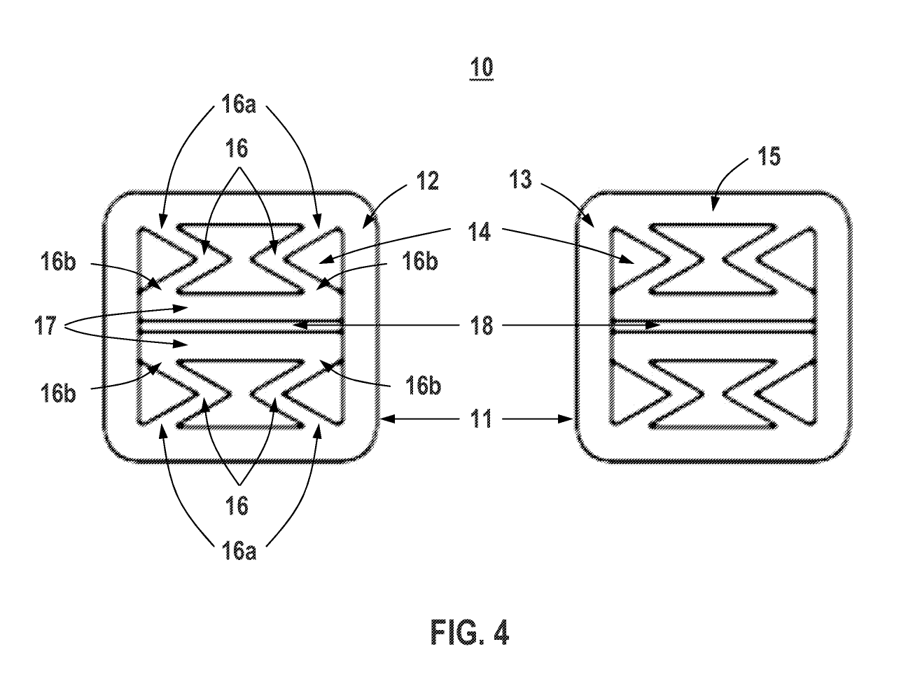

FIG. 4 illustrates a second exemplary embodiment of a device 10 for occlusion of skin tags 1 according to the present invention. Similar elements as shown in FIG. 3 are denoted by the same reference numerals in FIG. 4. The left side of FIG. 4 shows the upper side 12 of the device 10, and the right side of FIG. 4 shows the underside 13 of the device 10.

As shown in FIG. 4, each occlusion member 17, pressure member 16, and a portion of the base member 11 adjacent to the pressure member 16 may be formed in the shape of a “W”. As illustrated in FIG. 4, there are four “W” formations in the device 10. Based on the shape, size, thickness, material properties, and other characteristics of the pressure member 16 and occlusion member 17, each pressure member 16 and occlusion member 17 may elastically deform to apply an appropriate occlusion force to an enclosed skin tag 1.

The pressure members 16 and occlusion members 17 of the device 10 illustrated in FIG. 3 may apply an average force of 18 g+/−10 g. The pressure members 16 and occlusion members 17 of the device 10 illustrated in FIG. 4 may apply an average force of 75 g+/−34 g. Accordingly, the gap 18 between opposing occlusion members 17 may be configured to apply occlusion force sufficient to occlude blood flow to the skin tag 1 in the range of about 5 g to about 200 g, preferably about 8 g to about 109 g. Further, for both devices 10, the occlusion distance, i.e., the size of the gap 18 between opposing occlusion members 17, may be in the range of about 0.1 mm to about 4.0 mm, preferably about 0.2 mm to about 2.0 mm. The above ranges for sufficient occlusion force, and gap size configured to apply sufficient occlusion force, have been shown by experimental testing to be effective for skin tags 1 up to about 3 or 4 mm in diameter.

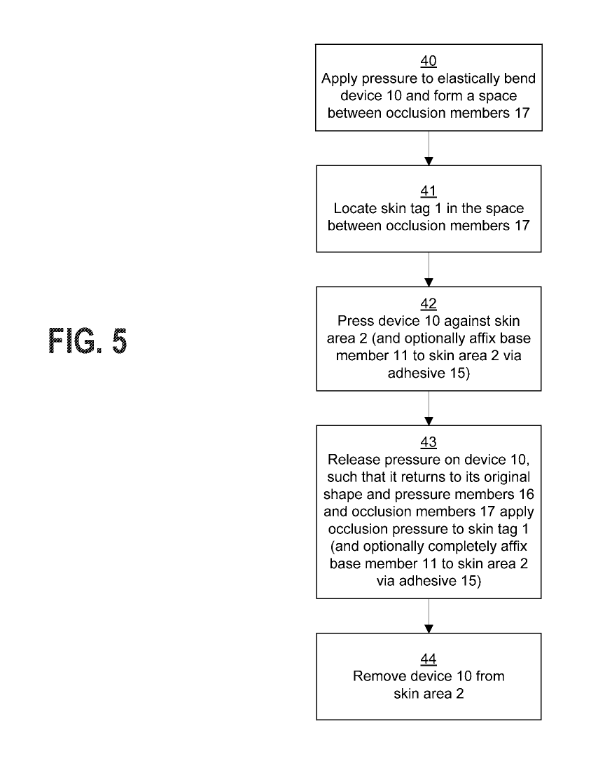

FIG. 5 illustrates a schematic flow diagram of a method of using a device 10 according to the present invention.

After a skin tag 1 is identified for occlusion by a patient or medical personnel, a device 10 according to the present invention may be chosen for application to the identified skin tag 1.

At step 40, the user may grasp the device 10 and apply pressure to the device 10, e.g., by holding the base member 11 between a thumb and a finger, for example, with one hand, such that the device 10 elastically bends away from the grasping thumb and finger and toward the identified skin tag 1. The elastic bending of the device 10 may allow the formation of a space greater than the gap 18 between the occlusion members 17 of the device 10.

At step 41, the user, while maintaining the elastically bent configuration of the device 10, may maneuver the device 10 such that the identified skin tag 1 is located in the enlarged space formed between the occlusion members 17.

At step 42, the user may press the device 10 against the skin area 2, and if the device 10 includes adhesive 15 on an underside 13, the adhesive 15 may then begin to affix the base member 11 to the skin area 2.

Then, at step 43, the user may release the pressure on the device 10, such that the device 10 returns to its original, e.g., flat, configuration, at which point the adhesive 15, if included, may completely affix the base member 11 to the skin area 2. Upon releasing at step 43 the manual pressure on the device 10, e.g., between the user's thumb and finger, the pressure members 16 and occlusion members 17 of the device 10 may apply occlusion force sufficient to occlude blood flow to the skin tag 1, preferably to a base of the skin tag 1.

At step 44, after the skin tag 1 has been fully occluded and is either ready to be removed from the skin area 2 or has already fallen off the skin area 2, the device 10 may be removed from the skin area 2.

The devices 10 as shown in FIGS. 3 and 4 may be made of plastic, or any other material that allows for flexion, can apply sufficient occlusion force, and can be manipulated according to the method of using a device 10 according to the present invention.

### Experimental Results

A clinical study of the use and outcome of the device according to the present invention was performed. In addition, a mixed bench top and clinical study of the relationship between compression distance, pressure and blood flow was also performed.

The devices used in the clinical study included both the “W” shaped and “Z” shaped devices illustrated in FIGS. 3 and 4. A bench study of the average force applied by the occlusion members and pressure members of each device was performed. For both designs, an occlusion distance of about 0.2 mm was chosen, similar to the gap between the occlusion members of the device when applied to a skin tag. The “W” shaped devices delivered an average force of 75 g+/−34 g, whereas the “Z” shaped devices delivered an average force of 18 g+/−10 g.

All potential subjects of the clinical study were assessed according to the following criteria. The inclusion criteria were: patients with skin tags who were interested in their removal, informed consent, and 30 years of age or older. The exclusion criteria were: other skin lesion(s) requiring medical treatment, past history of malignant melanoma or other condition motivating histopathological diagnosis of all treated lesions, pigmented base, younger than 30 years of age, and pregnant women.

Once the above criteria were met, lesions up to 6 mm in base were included in the study. The study was based on four visits. On Day 0, a visual inspection and selection of the study lesions took place. The size of the skin tags was determined and the form classified. Blood flow measurements using laser doppler, e.g., laser doppler perfusion imaging (LDPI), or laser doppler flowmetry (LDF), were performed before and directly after application of a device in order to allow for comparison of the grade of occlusion achieved and the outcome. On Day 2/3, the progress of the treatment was assessed by visual inspection and measurement of the blood flow using laser doppler. On Day 5/6, after visual inspection, the device was removed and the status of the treatment noted. The visit was completed by blood flow measurement using laser doppler. On Day 21-28, the final visit day, a final medical assessment was performed. The success of the treatment was graded by visual inspection.

The appearance of the lesion during treatment and at Day 21-28 follow up was documented according to the following possible outcomes:

(i) Grading of appearance of the skin tag at day 5/6 (removal): 0—skin-colored; 1—swollen; 2—dusky; 3—dry/necrotic; 4—came off with device; 5—came off with device and bled; 6—inflamed/bleeding.

(ii) Grading of appearance at day 21-28 (outcome): 0—skin-colored; 2—dusky; 4—came off; 7—came off with some residual left. Successful outcome was considered for gradings “4” and “7” at Day 21-28.

The study encompassed the treatment of 177 skin tags on 32 subjects, of whom 24 were women and 8 were men. The subjects' ages ranged between 36 and 78 years with an average age of 61 years. Of the 177 skin tags, 166 skin tags (94%) reached the planned device removal Day 5/6 and 160 skin tags (90%) reached the final assessment between Days 21 and 28 as described above.

A total of 17 lesions left the study prematurely. In 5 cases, the device was removed after initial measurements on Day 0 since base pigmentation was observed, thus fulfilling an exclusion criterion. Of the 172 skin lesions then remaining in the study, a total of 12 skin tags (7%) did not complete the protocol due to (1) patient not attending the follow up during the study (2 lesions); (2) skin tag treated by other means due to swelling at Day 2/3 or 5/6 (2 and 5 lesions, respectively); and (3) loosening of the device (3 lesions). Swelling occurred mostly for larger lesions which were more than 2 mm in base diameter and more than 5 mm long. The devices that loosened had been applied in the neck region.

The overall success of the treatment of skin tags (treatment outcome grading “4” and “7”) for the 172 skin tags was 65%.

The size of the skin tag showed a marked influence on the outcome of the treatment. Generally, successful treatment was highest for smaller tags. For skin tags with base diameter <1 mm, the success rate was 90%. For skin tags with a base diameter <2 mm, the success rate was 76%. These two groups constituted 83% of all skin tags treated in the study.

Concerning skin tag morphology, the best results were achieved for lamellar and filiform tags, illustrated in FIG. 1 (86% and 79% successful outcome, respectively). The success rate for other morphologies within this size class was slightly lower (64-76%). Most of the treated lesions were located in the patients' axillae and neck regions. A better outcome for skin tags localized to the axillae was observed compared to the neck (85% vs. 71%). The success rate decreased slightly with increasing age of the patient.

The relation between blood flow reduction in the region of interest corresponding to the skin tag and outcome was also analyzed. A decrease of blood flow was observed in 96.9% of lesions after device application. At Days 2/3, 87.6% showed some degree of occlusion, leading to successful outcome in 80.5% of these lesions. For skin tags for which no decrease in blood flow could be measured, only 37.5% of applications had a successful outcome.

A further analysis was performed to assess the impact of 20%, 40%, and 60% reduction in blood flow on the outcome. No significant increase in the rate of successful outcome was observed for higher flow reduction. Remarkably, the success rate for lesions not fulfilling the success criteria increased to 52%, 69%, and 71%, respectively. Thus, a measured decrease to any degree in blood flow after device application was important for a successful outcome rather than the degree of occlusion achieved.

Patients were also asked about their degree of discomfort at all visits. Discomfort or pain were noted on a visual analogue scale (VAS) graded from 0 (no discomfort) to 10 (highest possible discomfort). Throughout the study, subjects rated their discomfort as minimal. The application procedure was generally experienced as “pricking” to a small degree. During the days following the application of the device, some itching occurred, in several cases accompanied by slight scratching. The majority of subjects (90%) graded the discomfort during treatment between 0 and 1. No discomfort scores higher than 2 were recorded.

In summary, in this study, the devices according to the present invention were tested to treat skin tags in a setting that resembles the everyday clinical environment. The success rate in lesions with a base diameter <1 mm was very high (90%). Lesions <2 mm in base diameter had a success rate of 76%. At an individual lesion level, the physical criteria set for successful outcome were removal with little or no scarring and with minimal discomfort. The outcome of the study is therefore extremely satisfactory according to these criteria.

The impairment of blood flow in the skin tag may be central to the treatment. It is believed that no previous studies are available on the expected blood flow levels in skin tags or appropriate target levels for reduction to hinder blood flow and promote necrosis of the skin tag. The device can deliver adequate pressure to achieve blood flow impairment levels that were either total or near to total without mechanically crushing or cutting the tissue. Measurements were performed immediately after device application and at Days 2/3, i.e., at only two points in time during the 5-day application period. Since a proportion of lesions where no flow reduction was detected had a successful outcome, it may be inferred that an adequate blood flow reduction was achieved at some time point during the 5-day period for these lesions to achieve necrosis. Thus, occlusion of blood flow may be the sole pathoetiological hypothesized mechanism for the effectiveness of the treatment concept.

Another important feature may be tolerability. In the present protocol, the skin area around the skin tag was protected by a thin adhesive film onto which the device was applied. In most situations this gave good tolerability. Repetitive movement gave some irritation especially in the creases of the neck and the frontal aspect of the axillae (because of arm movement). If irritation in a lesion became pronounced in the present study, a decision was made to interrupt the study and remove the skin tag surgically. This decision was conservative and may have been unnecessary because a degree of inflammation may in fact promote vessel occlusion.

These experimental results demonstrate the effectiveness of the device for skin tag removal. The treatment is safe and well-tolerated with virtually no discomfort. In addition, the cosmetic results of the use of the device are excellent.

The foregoing description discloses only non-limiting embodiments of the present invention. Modification of the above-disclosed exemplary devices, and a method of using the same, which fall within the scope of the invention, will be readily apparent to those of ordinary skill in the art.

Accordingly, while the present invention has been disclosed in connection with the above non-limiting embodiments, it should be understood that other embodiments may fall within the spirit and scope of the invention, as defined by the following claims.

What is claimed is:

1. A device for occlusion of a skin tag projecting from a skin area, comprising:
a base member having an upper side, an underside, and an aperture for enclosing the skin tag;
a first pressure member connected to the base member at one end of the first pressure member;
a first occlusion member connected to an other end of the first pressure member for applying an occlusion force to the enclosed skin tag;
a second pressure member connected to the base member at one end of the second pressure member;
a second occlusion member connected to an other end of the second pressure member for applying the occlusion force to the enclosed skin tag; and
a gap between the first and second occlusion members configured to apply the occlusion force sufficient to occlude blood flow to the skin tag.

2. The device according to claim 1, wherein the underside of the base member includes an adhesive for fixing the base member to the skin area.

3. The device according to claim 1, wherein the base member is one of rectangular, square, circular, oval and elliptical.

4. The device according to claim 1, wherein the other end of the first pressure member faces the other end of the second pressure member.

5. The device according to claim 1, wherein the first occlusion member, the first pressure member, and a portion of the base member adjacent to the first pressure member are formed in the shape of one of a “Z” and a “W”.

6. The device according to claim 1, wherein the second occlusion member, the second pressure member, and a portion of the base member adjacent to the second pressure member are formed in the shape of one of a “Z” and a “W”.

7. The device according to claim 1, wherein a pair of first pressure members is connected to the first occlusion member.

8. The device according to claim 1, wherein a pair of second pressure members is connected to the second occlusion member.

9. The device according to claim 1, wherein the first pressure member includes a plurality of first pressure members, and the first occlusion member includes a plurality of first occlusion members.

10. The device according to claim 1, wherein the second pressure member includes a plurality of second pressure members, and the second occlusion member includes a plurality of second occlusion members.

11. The device according to claim 1, wherein the first occlusion member faces the second occlusion member for jointly applying the occlusion force to the enclosed skin tag.

12. The device according to claim 1, wherein the occlusion force sufficient to occlude blood flow to the skin tag is in a range of about 5 g to about 200 g.

13. The device according to claim 1, wherein the occlusion force sufficient to occlude blood flow to the skin tag is in a range of about 8 g to about 109 g.

14. The device according to claim 1, wherein the gap between the first and second occlusion members is in a range of about 0.1 mm to about 4.0 mm.

15. The device according to claim 1, wherein the gap between the first and second occlusion members is in a range of about 0.2 mm to about 2.0 mm.

16. The device according to claim 1, wherein the device is one of flat and disc-shaped.

17. A method of using a device for occlusion of a skin tag projecting from a skin area, the device comprising a base member having an upper side, an underside, and an aperture for enclosing the skin tag, a first pressure member connected to the base member at one end of the first pressure member, a first occlusion member connected to an other end of the first pressure member, a second pressure member connected to the base member at one end of the second pressure member, a second occlusion member connected to an other end of the second pressure member, and a gap between the first and second occlusion members configured to apply an occlusion force sufficient to occlude blood flow to the skin tag, the method comprising:
applying compression to the device, whereby the gap between the first and second occlusion members is enlarged;
enclosing the skin tag within the enlarged gap between the first and second occlusion members; and
releasing the compression on the device, whereby the occlusion force is applied to the skin tag via the first and second occlusion members and the first and second pressure members.

18. The method according to claim 17, wherein the compression is manually applied by a user's thumb and finger.

19. The method according to claim 17, wherein the device is applied with one hand of a user.

20. The method according to claim 17, further comprising:
adhering the base member to the skin area via an adhesive.

21. The method according to claim 17, wherein the occlusion force sufficient to occlude blood flow to the skin tag is in a range of about 5 g to about 200 g.

22. The method according to claim 17, wherein the occlusion force sufficient to occlude blood flow to the skin tag is in a range of about 8 g to about 109 g.
